(19) United States
(12) Patent Application Publication
(10) Pub. No.: US 2009/0326387 A1
(43) Pub. Date: Dec. 31, 2009

(54) Electrocardiogram and Respiration Monitoring in Animals

(75) Inventors: Andres Belalcazar, St. Paul, MN (US); Loell Boyce Moon, Ham Lake, MN (US); Paul A. Haefner, Circle Pines, MN (US); Scott R. Tiesma, New Hope, MN (US)

Correspondence Address: FISH & RICHARDSON P.C., P.O. BOX 1022, MINNEAPOLIS, MN, 55440-1022, US

(73) Assignee: Transoma Medical, Inc.

(21) Appl. No.: 12/165,415

(22) Filed: Jun. 30, 2008

(51) Int. Cl. A61B 5/02

(52) U.S. Cl. 600/484

(57) ABSTRACT

An ambulatory animal monitoring system includes a wearable structure constructed to be worn about a body of a non-human animal to be monitored. The system includes a plurality of electrical signal conduits each associated with the wearable structure and each connectable to a different one of a plurality of surface electrode components. The system includes processing and control device adapted to be worn with the wearable structure, the processing and control device comprising a) an ECG monitoring component and b) an impedance level monitoring component that generates data indicative of electrical impedance levels of the animal over time.

### BACKGROUND

An ECG is a recording of electrical activity of a heart of a subject over time. ECG devices generally measure the potential differences between various selected points on the body of a test subject. The measured potential differences may mirror the electronic activities of the heart muscle and provide some insight into the cardiac health of the test subject. The ECG measurement can be performed by way of attaching electrodes to the body of a test subject in predetermined places. For example, the electrodes can be placed around the heart at various points on the test subject.

The placement of the electrodes on the test subject may require experience and anatomical knowledge about the test subject. In the event that the test subject is a small animal, it may be difficult to ascertain the proper anatomical positions for electrode placement. As a result, electrodes may be improperly positioned which may provide skewed test results. In addition, electrodes may lose electrical conductivity over time. These electrodes may cause excessive electrical noise due to disruptions of electrical conductivity which may lead to difficulty in interpreting received measurement data.

Respiration monitoring has been performed using respiratory inductive plethysmography (RIP) technology. RIP bands can be placed around the torso of a subject such that when the subject respires, the RIP bands inductively register a change in the shape of the torso. For example, the RIP bands can include an expandable, serpentine-shaped conductor that encircles the subject's torso, and the change can be detected by measuring how the inductance of the conductor changes with the subject's breathing.

### SUMMARY

The invention relates to improved ECG and respiration monitoring in animals. Respiration monitoring can be performed by measuring an impedance value of the animal's body concurrently with detection of ECG signals. A system for animal monitoring can perform measurements while permitting the animal to ambulate and not be tethered to stationary equipment. Respiration monitoring can be performed in more than one channel, for example to permit both thoracic and abdominal respiration to be detected. ECG monitoring can be performed using a surface electrode that is also used for respiration monitoring.

In a first aspect, an ambulatory animal monitoring system includes a wearable structure constructed to be worn about a body of a non-human animal to be monitored. The system includes a plurality of electrical signal conduits each associated with the wearable structure and each connectable to a different one of a plurality of surface electrode components. The system includes processing and control device adapted to be worn with the wearable structure, the processing and control device comprising a) an ECG monitoring component that generates data indicative of an ECG signal over time by measuring the ECG signal of the animal using surface electrode components connected to the plurality of electrical signal conduits; and b) an impedance level monitoring component that generates data indicative of electrical impedance levels of the animal over time by i) injecting current between at least two surface electrode components connected to the plurality of electrical signal conduits, and ii) measuring a resulting voltage level between at least two surface electrode components connected to the plurality of electrical signal conduits, at least one of the surface electrode components between which the current is injected not being used for the measuring of the resulting voltage.

Implementations can include one or more of the following features. The plurality of electrical signal conduits can include at least three electrical signal conduits. At least one surface electrode component whose output is connected to the plurality of electrical signal conduits can be used both in measuring the ECG signal and in measuring the electrical impedance level. The impedance measurement circuitry can include two channels of impedance measurement circuitry for measuring over time an electrical impedance level between two different sets of surface electrode components whose outputs are connected to the plurality of electrical signal conduits, so as to measure over time an electrical impedance level across two different portions of the animal's body. The plurality of electrical signal conduits can include at least six electrical signal conduits (ESC1 through ESC6) for using at least six surface electrode components (SEC1 through SEC6) with the animal monitoring system. At least electrical signal conduits ESC1, ESC2, ESC4 and ESC5 can be used for electrical impedance measurements; and at least electrical signal conduits ESC2 and ESC4 are used for ECG measurement. Electrical signal conduits ESC3 and ESC 6 can also be used for impedance measurements, with electrical signal conduits ESC1, ESC2, ESC4 and ESC5 being used for a first channel of electrical impedance measurements, and electrical signal conduits ESC2, ESC3, ESC5 and ESC6 being used for a second channel of electrical impedance measurements. The animal monitoring system can further include a telemetry component adapted to be worn with the wearable structure, the telemetry component for wirelessly communicating to another device the generated data indicative of an ECG signal over time and the generated data indicative of electrical impedance levels of the animal over time. The system can be configured such that when the system is placed on an animal to be monitored, the system does not restrict ambulatory movement of the animal by way of tethering the animal to stationary equipment. The processing and control device can be adapted to determine from the electrical impedance measures whether a placement of an electrode component on the animal is unsatisfactory. The processing and control device can further be adapted to perform the ECG sensing and the impedance measuring using electrode components other than an electrode component whose placement has determined to be unsatisfactory. The processing and control device can further be adapted to produce a signal indicative of an electrode component having an unsatifactory placement. The impedance level monitoring component can generate the data indicative of electrical impedance levels such that the data is configured to be used in monitoring animal respiration.

In a second aspect, an animal monitoring system includes wearable componentry comprising: a wearable structure constructed to be worn about a body of a non-human animal to be monitored; a plurality of electrical signal conduits each associated with the wearable structure and connectable to a different one of a plurality of surface electrode components; processing and control apparatus adapted to be worn with the wearable structure, the processing and control apparatus comprising a) an ECG monitoring component that generates data indicative of an ECG signal over time by measuring the ECG signal of the animal using surface electrode components connected to the plurality of electrical signal conduits; and b) an impedance level monitoring component that generates data indicative of electrical impedance levels of the animal over time by i) injecting current between at least two surface electrode components connected to the plurality of electrical signal conduits, and ii) measuring a resulting voltage level between at least two surface electrode components connected to the plurality of electrical signal conduits, at least one of the surface electrode components between which the current is injected not being used for the measuring of the resulting voltage; and a telemetry component adapted to be worn with the wearable structure, the telemetry component for wirelessly communicating the generated data indicative of an ECG signal over time and the generated data indicative of electrical impedance levels of the animal over time. The system includes receiving and processing apparatus comprising a wireless receiver that receives the generated data wirelessly transmitted from the telemetry component of the wearable componentry, and an analysis module for analyzing ECG and respiration of the animal based on the generated data.

Implementations may provide one or more of the following advantages. An animal monitoring system can be provided for improved ECG and respiratory monitoring while permitting the animal to ambulate. Respiratory monitoring can be performed based on an impedance measured through at least part of an animal's body. ECG and respiratory monitoring can be performed using at least one common electrode. An animal monitoring system can be provided that can detect unsatisfactory operation of a surface electrode and instead perform the monitoring using another electrode. Additionally, the monitoring system could provide notification that one or more electrodes are exhibiting poor conductivity and require attention or replacement.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of an example ambulatory animal monitoring system shown fitted on a canine laboratory animal.

FIG. 2 illustrates example electrode positions for monitoring both thoracic and abdominal impedances and ECG signals in a canine laboratory animal.

FIG. 3 is a block diagram of an example of an ambulatory animal monitoring system.

FIG. 4 is an example of an excitation pulse that can be used in the systems of FIGS. 1-3.

FIG. 5 is a flow diagram of an example method of monitoring an animal.

FIGS. 5A-B provide an anatomical reference for an example laboratory animal.

FIGS. 6A-D illustrate example electrode configurations.

Like reference symbols in the various drawings indicate like elements.

### DETAILED DESCRIPTION

An important aspect of animal management is to monitor the animal's physical condition. Respiration is a significant indicator of the animal's well-being. For example, an unusually heavy or weak breathing can be a symptom that the animal is in a particular physical condition. Likewise, if the animal's breathing changes—say, from normal to abnormal—this can also be an important indication for those monitoring the animal. Other signs of physical condition, including ECG data, can also be important. Thus, it is vital to be able to monitor relevant indications of the animal's physical condition. The present disclosure describes examples of systems and techniques that can provide improved monitoring of animals.

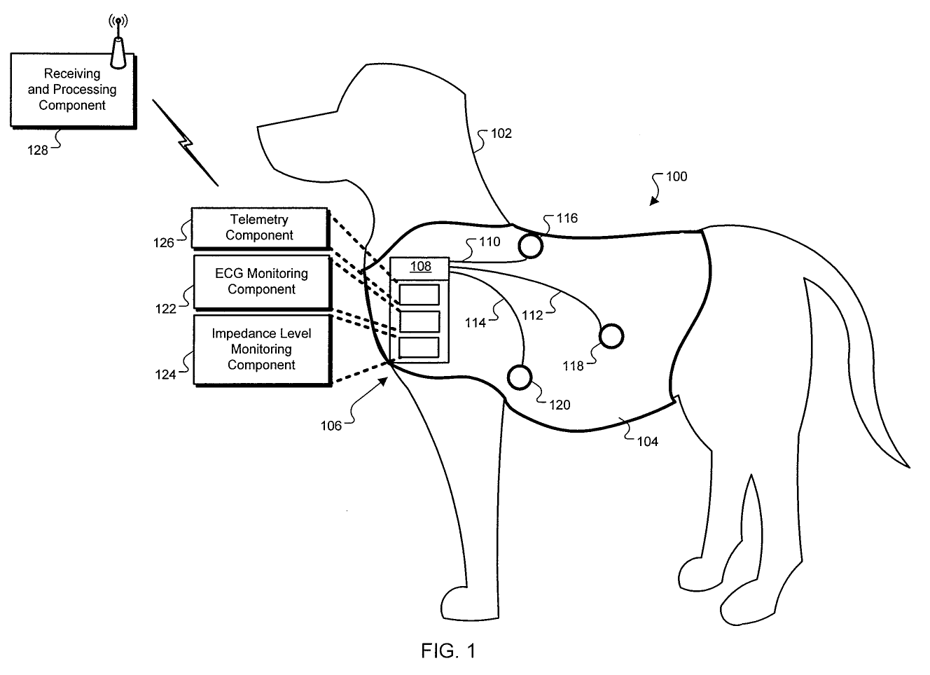

During preclinical testing of pharmaceutical compounds on animal subjects, various physiological parameters of the animal subjects can be monitored using an animal monitoring system 100 shown in FIG. 1. In particular, the system 100 can monitor physiological parameters of the animal while the animal is ambulatory and free from tethered wiring, tubing, or other encumbering equipment. In some implementations, the animal monitoring system 100 can be used to acquire and analyze both ECG data and time varying impedance values in the animal's body. The ECG data and impedance level data can be acquired non-invasively using two or more electrode devices placed on the skin surface of the animal subject. The electrodes can be used to sense ECG signals and impedance levels of the animal. More specifically, time-varying thoracic impedance values, time-varying abdominal impedance values, and ECG values can be obtained from the electrode devices concurrently, successively, or in an overlapping manner. In some implementations, further analysis can be performed on the obtained data to ascertain values for tidal volume, respiratory rate, inspiratory time or interval and flow, and expiratory time or interval and flow. In other implementations, other sensors may be included in the animal monitoring system 100.

FIG. 1 is a diagram of the example ambulatory animal monitoring system 100 shown fitted on a canine laboratory animal 102. In one implementation, the system 100 includes a wearable structure, such as a jacket 104, that is constructed to be worn about the torso or body of the canine 102 to facilitate monitoring functions. For example, the jacket 104 is shown placed on the canine 102 in such a fashion that the jacket 104 protects or shields electrode(s) on the animal while not restricting ambulatory movement of the canine by way of tethering the animal to stationary equipment. Although FIG. 1 illustrates the animal monitoring system 100 placed on a canine animal, other animals can be fitted with the system 100 for monitoring purposes. Other configurations of the system 100 can be used.

The depicted example jacket 104 includes a processing and control device 106 that may be adapted to be worn with, attached to, or otherwise fastened to the jacket 104. For example, the processing and control device 106 may be placed within a pocket or sleeve on the jacket 104 or alternatively may be affixed to the jacket, such as with hook-and-loop fastener, tape, or any other fastener. The processing and control device 106 can, for example, be used to inject current to the canine 102 via surface electrodes and measure signals received at one or more of the surface electrodes.

The processing and control device 106 here includes a wiring system 108 that can include any number of electrical signal conduits for use in the monitoring. For example, the conduit(s) can allow disposable surface electrodes to be removably connected to the device 106. The illustrated system 100 here includes three electrical wires 110, 112, and 114 which are detachably connected to the wiring system 108. In one example, the electrical wires 110, 112, and 114 may be combined in an electrical harness constructed for attachment to the wiring system 108. The wire harness (110-114) and surface electrodes (116-118) can be attached within the wiring system 108 to electrical signal conduits. The connection to the electrical signal conduits may be provided by a single connector or separate connectors for each wire. Any kind of electrical connector can be used, such as a plug. In some implementations, the electrical wires 110-114 can be fixably attached to wiring system 108 and disposable electrodes can connect at the ends of the wires 110-114.

In some implementations, each wire 110, 112, and 114 may be connectable to a different surface electrode component. As shown in FIG. 1, the wires 110, 112, and 114 are connected, respectively, to surface electrode components 116, 118, and 120. In some implementations, the electrical wires 110, 112, and 114 and the surface electrode components 116, 118, and 120 can be permanently attached to each other, respectively, and may therefore provide pluggable component(s) to wiring system 108.

The processing and control device 106 in this implementation includes an ECG monitoring component 122, an impedance level (IL) monitoring component 124, and a telemetry component 126. In one example implementation, the components 122, 124, and 126 can be included in one packaged device that can be held by the jacket 104. In some implementations, the ECG monitoring component 122 and the IL monitoring component 124 may be placed in one area of the jacket 104, while the telemetry component 126 can be placed elsewhere to, for example, facilitate wireless access to the jacket 104 and/or decrease interference noise that may be caused when operating components 122 and 124 in the vicinity of a wireless transceiver. The ECG monitoring component 122 and the IL monitoring component 124 cab be provided as separate components or integrated into a common component.

In general, the ECG monitoring component 122 can generate data indicative of an ECG signal over time by measuring the ECG signal of the animal using two or more surface electrode components connected to respective electrical signal conduits. For example, the ECG monitoring component 122 can measure the electrical potential between the surface electrode 116 and the surface electrode 120 to provide a differential bio-potential signal. In some implementations, signals on both electrodes 116 and 120 may be measured at some frequency to determine the ECG signal over time.

The IL monitoring component 124 generates data indicative of electrical impedance levels in the animal 102 over time. In some implementations, the IL monitoring component 124 can inject a current between two or more surface electrodes connected to a number of electrical signal conduits. For example, the IL monitoring component 124 can inject current between surface electrodes 116 and 118, between surface electrodes 118 and 120, or between surface electrodes 116 and 120 shown in FIG. 1. In some implementations, ECG and impedance can be measured using two surface electrodes, such as the surface electrodes 116 and 118. Below will be described an example that involves six surface electrodes. Accordingly, fewer or more surface electrodes than in the present example can be used to monitor physiological parameters, such as ECG and impedance levels in an animal.

The IL monitoring component 124 can measure a resulting voltage level which occurs between surface electrode components as a result of the current being injected. For example, if the IL monitoring component 124 injects current between surface electrodes 116 and 118, any of the surface electrodes, such as the surface electrode 118, can measure the resulting voltage.

In some implementations, various parameters in components 122 and 124 may be adjustable or programmable, either automatically (e.g., using signals transmitted from the receiver and corresponding processing circuitry 128) or manually (e.g., accessing the device 122 or 124 in the jacket 104 and entering parameters). In one example, the gain for individual electrode sensors may be adjustable (e.g., manually, or automatically) to facilitate a high signal-to-noise ratio in a variety of operating environments. In some implementations, frequency and current amplitude are both adjustable to maximize the signal-to-noise ratio while minimizing power consumption.

The telemetry component 126 can be adapted to be worn with the jacket 104 for wirelessly communicating generated ECG data and/or impedance level data. For example, the telemetry component 126 can send data to a receiving and processing component 128 located elsewhere, such as in a laboratory or a medical facility where the animal is being monitored. In some implementations, the ECG data and impedance level data can be sent to component 128 substantially in real time. In other implementations, the processing and control device 106 can record incoming data over a particular time period and provide the data via upload at a later time. In such implementations, the telemetry component 126 can be omitted from the device 106.

The receiving and processing component 128 may receive ECG data, impedance level data, and other physiological parameters sensed in canine animal 102 and further process the data. The receiving and processing component 128 here includes a wireless receiver that receives generated data wirelessly from the processing and control device 106. The receiving and processing component 128 can convert detected voltages into an impedance (e.g., by dividing the magnitude of the detected voltage by the magnitude of the current signal). In some implementations, the receiving and processing component 128 may receive raw data from the animal monitoring system 100, and the raw data could be appropriately filtered and processed. For example, the measured voltage (e.g., voltage sensed by surface electrodes 116, 118, and/or 120) could be demodulated based on a magnitude of an applied current (e.g., the current applied by the impedance level monitoring component 124) to determine instantaneous impedance values.

In some implementations, the animal monitoring system 100 can use one or more surface electrodes 116, 118, or 120 connected to corresponding conduits 110, 112, and 118 to measure and/or sample the ECG signal while additionally measuring the electrical impedance levels. In some implementations, the ECG signals are captured at substantially the same time that impedance values are obtained (e.g., signals on the appropriate electrodes may be sampled at some frequency, and the samples may alternate between sampling impedance information (e.g., voltage induced by the above-described current injection) and sampling ECG information (e.g., each sample or based on some other pattern, such as one impedance sample for every five ECG samples). In such implementations, the ECG (or other bio-potential information) may be sampled in a manner that is synchronized with the injected current signal (e.g., such that the sample is made when the IL monitoring component 124 is not actively providing current to the animal tissue, such as the off portion of a pulsed current signal). In other implementations, the electrical signal conduits (110-114) and/or the surface electrodes (116-120) may be used for either capturing ECG or other bio-potential information, or for capturing thoracic impedance information or abdominal impedance information, and the current injected into the electrodes may be remotely programmable or adjustable. Other configurations and measurements can be used. For example, all three surface electrodes 116, 118, and 120 in the triangular configuration shown in FIG. 1 could be employed to capture bio-potential information.

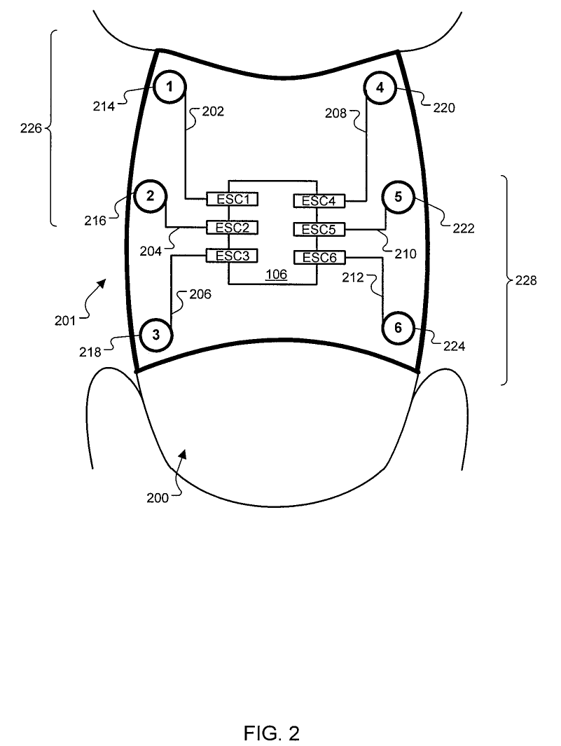

FIG. 2 illustrates example electrode positions for monitoring both thoracic and abdominal impedances and ECG signals in a canine laboratory animal 200. In some implementations, measuring both thoracic and abdominal impedances can be performed using three or more electrodes. If only one impedance (e.g., either thoracic or abdominal) is to be measured, two or more electrodes can be employed to perform the measurement.

In this example, an animal monitoring system 201 is shown having six electrical signal conduits including ESC1, ESC2, ESC3, ESC4, ESC5, and ESC6, schematically illustrated as connectively coupled to the processing and control device 106. ESC1 to ESC6 are additionally shown connectively coupled to six surface electrode components SEC1, SEC2, SEC3, SEC4, SEC5, and SEC6 (214-224) via respective wires 202 through 212. The electrical conduits ESC1 to ESC6 can be provided in the animal monitoring system 201 for use with electrodes SEC1 to SEC6 (214-224). That is, the outputs of the surface electrode components 214-224 can be connected to one or more electrical signal conduits ESC1 to ESC6 such that one or more electrical impedance level measurements can be taken across a thoracic region 226 as well as an abdominal region 228.

Strategically placing the surface electrodes SEC1 to SEC6 (214-224) across the thoracic region 226 and the abdominal region 228 may provide two channels of impedance measurement. In some implementations, each region 226 and 228 may be provided as separate impedance measurement circuitry. In other implementations, a combination of impedance measurement circuitry can be constructed across the regions 226 and 228.

As shown in FIG. 2, the electrodes SEC1 (214), SEC2 (216), SEC4 (220), and SEC5 (222) may be configured to detect signal in the thoracic region 226. Similarly, the electrodes SEC2 (216), SEC5 (222), SEC3 (218), and SEC6 (224) may be configured to detect signal in the abdominal region 228. In general, multiple combinations of conduits ESC1 to ESC6 can be used for measuring over time an electrical impedance level with two different sets of surface electrode components across two different regions (e.g., thoracic region 226 or abdominal region 228) of the animal's torso. For example, it can be useful to determine the extent to which the animal breathes using the thoracic region 226 and the abdominal region 228, respectively. Embodiments of the present systems and techniques can therefore advantageously monitor both these aspects of respiration. Further, any of the conduits ESC1 through ESC6 may be used to measure and/or sample an ECG signal in animal 200.

As an example, the animal monitoring system 201 can employ ESC1, ESC2, ESC4, and ESC5 to provide electrical impedance measurements, while utilizing ESC2 and ESC4 for ECG measurements. In general, the measurements for ECG and impedance levels can be performed concurrently, successively, or in an overlapping manner. The conduits ESC1, ESC2, ESC4, and ESC5 may, for example, be used to monitor thoracic respiration using tetra-polar impedance measurements.

In another example, the animal monitoring system 201 can employ electrical signal conduits ESC3 and ESC6 to measure impedance levels while the electrical signal conduits ESC1, ESC2, ESC4, and ESC5 can be used for a first channel (e.g., thoracic region 226) of electrical impedance measurements. Namely, ESC1, ESC2, ESC4, and ESC5 can be used to monitor thoracic respiration. In addition, the electrical signal conduits ESC2, ESC3, ESC5, and ESC6 can be used for a second channel (e.g., abdominal region 228) of electrical impedance measurements. That is, ESC2, ESC3, ESC5, and ESC6 can be used to monitor abdominal respiration. In addition to monitoring both thoracic respiration and abdominal respiration, ESC4 and ESC2 combined with one other conduit (e.g., functioning as a ground) can monitor ECG signals.

While the electrodes SEC1 to SEC6 (214-224) are shown horizontally aligned on animal 200, other placement configurations are possible. For example, if the system 201 were used on a smaller animal, the electrodes can be dispersed having four electrodes in the thoracic area and four separate electrodes in the abdominal area.

In some implementations, fewer than six electrodes can be used to measure impedance levels and ECG signals in the animal 200. For example, a current may be injected between SEC1 (214) and SEC2 (216) to measure the impedance level of the thoracic region 226. In a similar manner, a current may be injected between SEC2 (216) and SEC3 (218) to measure the impedance level of the abdominal region 228. Continuing with the above example and assuming the use of the same three electrodes, the ECG signal can be measured between any two of the three electrodes SEC1 (218), SEC2 (216), or SEC3 (218).

In some implementations, the electronic processing and control device 106 can be adapted to determine whether a placement of an electrode component on the animal 200 is unsatisfactory. For example, a particular conduit (e.g., any of conduits ESC1 through ESC2) may return an impedance measurement value that does not correspond to other measurements taken by other sensors within the same timeframe and/or experiment. That is, the electronic processing and control device 106 can determine whether a conduit has become detached from the animal, stripped out of the harness, or otherwise been compromised. Accordingly, the electronic control and processing device 106 may be adapted to perform the intended ECG sensing and the impedance measuring using another (e.g., non-failing) electrode component. In some implementations, the ECG sensing and the impedance measurements can be taken on multiple electrodes to ensure the results are verifiably accurate. In some implementations, the electronic processing and control device 106 can be adapted to produce a signal indicative of an electrode component having an unsatifactory placement. For example, the device 106 may emit an alarm such as an audible indicator, a visual indicator, a wirelessly transmitted email or other type of notification indicating an electrode, conduit, or wire is failing and/or inaccurate. In some implementations, an alarm may provide an indication to laboratory staff to replace one or more electrodes.

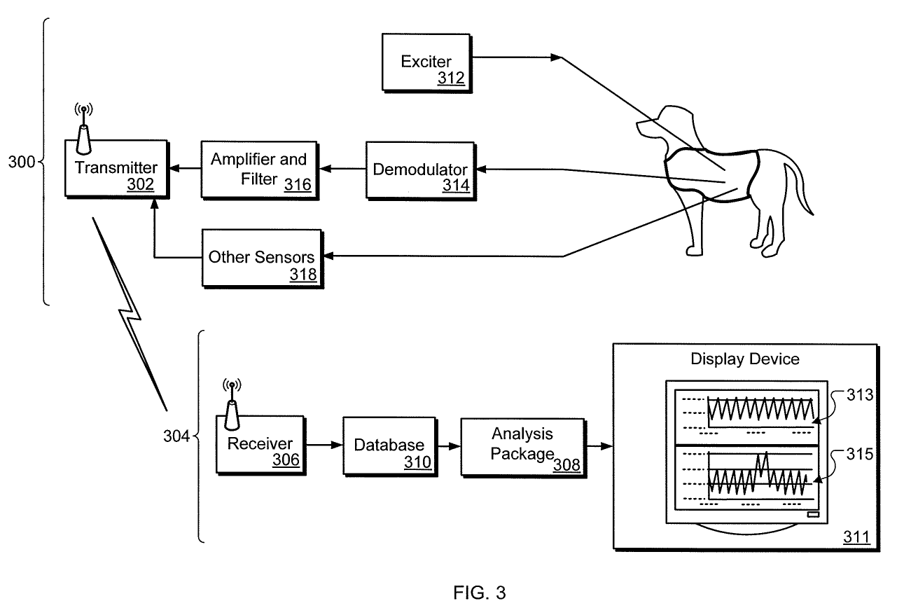

FIG. 3 is a block diagram of an example of an ambulatory animal monitoring system 300. In general, the system 300 includes an animal monitoring system integrated with wireless communication circuitry. More particularly, the animal monitoring system 300 includes a wearable structure constructed to be worn about a body of a non-human animal to be monitored. The system 300 includes a telemetry component adapted to be worn with the wearable structure. In this example, the telemetry component is shown as transmitter 302. The transmitter 302 may be used to wirelessly communicate data generated by system 300 (e.g., ECG signal data and electrical impedance levels) to a receiving and processing apparatus, such as an external receiver portion 304. The receiver portion 304 may include a wireless receiver 306 that receives data generated in system 300 and wirelessly transmits the data from the transmitter 302. In addition, the receiver portion 304 includes an analysis package 308 for analyzing ECG and respiration data measured in a monitored animal. The analysis package 308 may have access to various external database structures, such as database 310, to receive uploaded or transmitted sensor data, for example. In some implementations, the telemetry component 126 (FIG. 1) can include at least the transmitter 302, and the receiving and processing component 128 can include at least the receiver portion 304, respectively.

The receiver portion 304 here also includes a display device 311. The display device 311 is an output device, such as a computer monitor for example, used to present information to a user. The display device 311 may present various animal measurements, including ECG signals 313 and respiratory data 315. The ECG signals 313 and respiratory data 315 may be presented graphically, as shown in display 311, textually, or embedded in another formatting scheme (e.g., a database, a website, etc). In some implementations, other sensor data retrieved from system 300 can be presented on display 311, such as an indication of whether the animal is lying down or moving. In some implementations, data may be presented on display 311 as raw numeric data sensed from system 300. For example, raw numeric data stored in database 310 can be viewed on display device 311. In another example, data can be presented on display 311 real time as sensed by system 300.

In some implementations, display device 311 may be a touchscreen device with menus for saving, printing, zooming, or otherwise manipulating display output. For example, users can enter tactile feedback into device 311 to manipulate waveforms, databases, or other data. For example, the ECG signal waveform 313 may be selected and scanned or scrolled laterally to view a broader sampling of data. In one example, the manipulated data can be used for future comparison to various other monitoring results.

The animal monitoring system 300 includes exciter circuitry 312. Exciter circuitry 312 may provide excitation pulses to system 300. The excitation pulses provide an injected current between two or more surface electrodes, for example, and upon receiving the current, one or more electrodes may perform ECG measurements and thoracic and/or abdominal impedance level measurements in an animal.

The animal monitoring system 300 also includes conditioning circuitry for conditioning ECG signals and impedance signals sensed by electrodes (not shown) in system 300. In the depicted example, the conditioning circuitry includes demodulator circuitry 314 and amplifier and filtering circuitry 316. In some implementations, a detected voltage (e.g., from an electrode) can be demodulated by the circuitry 314 to form a time-varying impedance signal. In some implementations, the voltage signal can be demodulated within the animal monitoring system 300, and a time-ordered sequence of impedance values can be transmitted to the external system 304 (e.g., using transmitter 302). In other implementations, the voltage signal can be directly transmitted (e.g., using transmitter 302), and the voltage signal can be demodulated in the external system 304 (e.g., based on a transmitted time-ordered sequence of magnitude values corresponding to the current signal, or based on a fixed magnitude of current stored in the external system 304. In some implementations, at least the amplifier and filter 316 can be included in the component 106 (FIG. 1). Other conditioning circuit configurations are possible.

The amplifier and filtering circuitry 316 may detect a voltage signal between two or more electrodes and amplify or filter the signal in a suitable fashion. In some implementations, the circuitry 316 may be used to amplify signals from various sensors. In some implementations, filtering may be applied in combination to individual or multiple signals received from any electrode in system 300. In one example, the circuitry 316 may include a filter (not explicitly shown) to filter ECG signals captured by a bio-potential sensor output of a signal that is captured by a thoracic impedance electrode. In some implementations, data can be removed through the application of various kinds of filters. For example, digital or analog filters can be applied to remove undesirable data. The filter can be, for example, a linear, non-linear, histogram-based, or any other appropriate type of filter. Alternatively, other forms of signal processing (e.g., forms of signal processing that are not traditionally characterized as filtering) can be applied to the data to remove particular portions or qualities of the data.

As shown in FIG. 3, the animal monitoring system 300 here also includes other sensors 318. For example, system 300 may include an accelerometer sensor to detect both posture and behavior or activity levels of an animal. Data from the accelerometer sensor can be used to remove windows of impedance data corresponding to posture or activity of the test subject that may be likely to cause corruption of impedance data. As a particular example, impedance data may be corrupted by vigorous activity of the animal (e.g., running) and an accelerometer can be employed to detect such vigorous activity. Based on detection by the accelerometer of the vigorous activity, corresponding impedance data can be removed-either before the data is sent or in an external system after data is sent.

As another example, the sensors 318 can include an electromyogram (EMG) sensor that can be employed to detect specific movements that may have a tendency to corrupt impedance data (e.g., certain movements of the front legs, in the case of a quadruped). In a similar manner as described above, impedance data that corresponds to EMG sensor-detected movements that are likely to corrupt the impedance data can be removed.

In some implementations, the animal monitoring system 300 may include onboard memory to, for example, store animal data (animal number, weight, etc.), ECG data, impedance data, configuration data, calibration data, experiment results, and other data. The memory may include all forms of non volatile memory, media and memory devices, including by way of example semiconductor memory devices, e.g., EPROM, EEPROM, and flash memory devices; magnetic disks, e.g., internal hard disks or removable disks; magneto optical disks. The memory can be supplemented by, or incorporated in, special purpose logic circuitry.

In operation, the wearable system 300 can receive an excitation pulse from excitation circuitry 312 and perform one or more impedance level measurements within an abdominal region or a thoracic region. In addition, the system 300 can measure ECG signals before, during, or after the excitation pulse is received. The measured signals can be demodulated, amplified, filtered, or otherwise conditioned in demodulator circuitry 314 and/or amplifier and filter circuitry 316. In tandem or concurrently, other sensor measurements can be performed in system 300. The conditioned measurements and other sensor data may be wirelessly transmitted (e.g., by transmitter 302) to the external system 304 and received at receiver 306. The received data may be stored in a database 310 and analyzed by the analysis package 308. In some implementations, the external system 304 may utilize a display device 311 to present analyzed data to a user.

The analysis package 308 may include modules for analyzing ECG data to provide R-wave, T-wave data, P-wave data, T-wave data, or other cardiac data. In some implementations, the analysis package 308 can analyze ECG data, and identify individual ECG and/or cardiac events (e.g., the location of T-waves, the location of P-waves, the location of the QRS complex, and the like) within the ECG data. In some implementations, the analysis package 308 can analyze impedance data to determine the efficacy of a particular pharmaceutical drug on lung function, for example. Other analysis tasks can be performed.

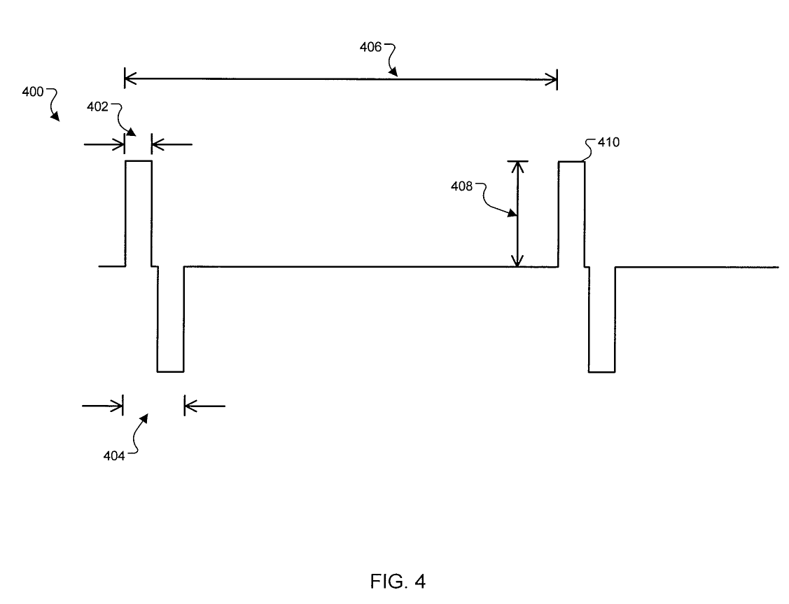

FIG. 4 is an example of an excitation pulse 400 that can be used in one or more implementations, such as in the system of FIG. 3. The excitation pulse 400 shown here is a square wave. In some implementations, the excitation pulse current may instead be continuously supplied in another form, such as a sinusoidal or triangular waveform.

The excitation pulse 400 generally delivers a periodic excitation signal to the one or more electrodes used in the animal monitoring systems shown in FIGS. 1-3. The excitation signal periodically injects current into one or more electrodes on an animal undergoing monitoring. As is typical, the pulse train 400 includes a pulse width 402, a period 404, a sample period 406, and an amplitude 408. The excitation pulse 400 can be provided with an excitation frequency from about 10 kHz to 100 kHz and a sample frequency from about 10 Hz to 60 Hz, for example.

During operation, one or more voltage levels between surface electrodes can be measured near the edge 410. The measurement taken near edge 410 can be a thoracic or abdominal impedance level measurement for the animal undergoing monitoring, within system 100, for example. More specifically, the excitation pulse 400 can provide a current signal between two electrodes (e.g., 116 and 118) and can detect a corresponding voltage between the two electrodes 116 and 118 that is modulated by an impedance (e.g., a thoracic impedance or an abdominal impedance) between the electrodes 116 and 118.

In some implementations, an ECG measurement can be time multiplexed such that it is performed before or after the excitation pulse is sent. In other implementations, an ECG measurement can be performed during the sending of the pulse on a different electrode, for example.

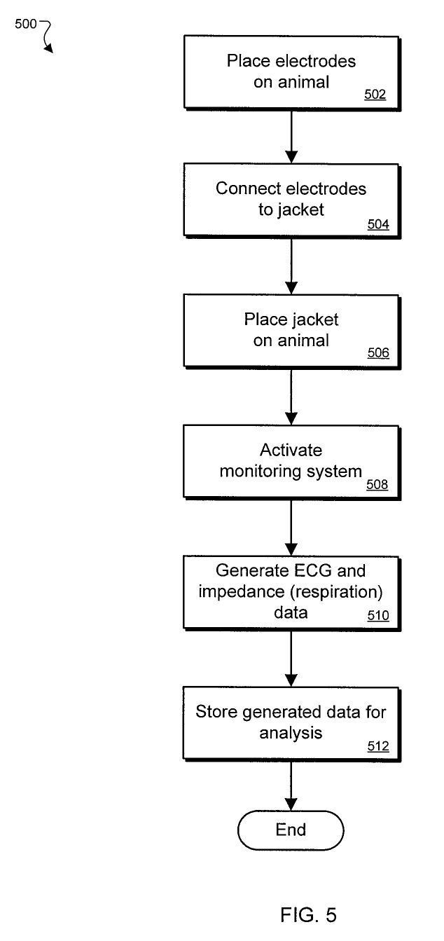

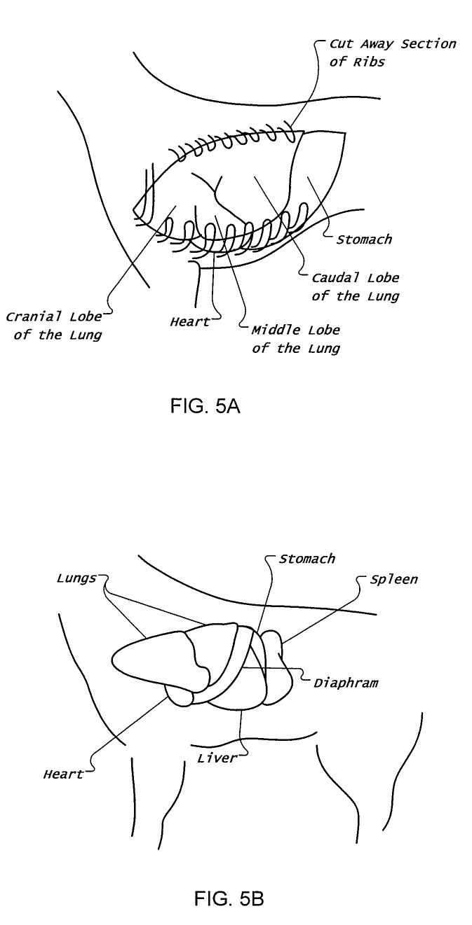

FIG. 5 is a flow diagram of an example method 500 of monitoring an animal. The method 500 can include placing (502) electrodes on an animal. For example, the animal 102 (FIG. 1) can be outfitted with one or more electrodes (116, 118, or 120). The electrodes 116-120 may be removably connected to the skin of the animal 102.

The method 500 can include connecting (504) electrodes to a jacket. For example, the electrodes 116-120 may be connected to the jacket 104 via wires 110-114. The wires 110-114 can be further connected to the wiring system 108 housing conduits. After connecting the electrodes, the method 500 can include placing (506) the jacket about the body of the animal. Placing the jacket over the connected electrodes may protect or shield electrode(s) while not restricting ambulatory movement of the animal 102.

The method 500 can include activating (508) the monitoring system, such as the systems 100, 201 or 300, for example. In one example, activating (508) monitoring systems may include uploading configuration parameters, enabling wireless communication, engaging conduits with electrodes, system calibrations, etc. In some implementations, the activation may include calibrating sensors. In other implementations, the activation (508) may include configuring an external system, such as the receiving and processing component 128, for receiving real time updates from one or more animal monitoring systems. For example, one external system may receive data from several functioning animal monitoring systems within a laboratory.

The method 500 can include generating (510) ECG and impedance (respiration) data. The data can be generated while a monitored animal is ambulatory. In some implementations, physical tests can be administered to determine heart effects, lung effects, or other respiration related changes detectable through electrodes on the animal monitoring system 100. The physical tests can be used, for example, to provide insight about how a particular animal may react to an administered drug.

The method 500 can include storing (512) generated data for analysis. For example, the generated data can be stored as raw data until further processing can be performed on the data. In some implementations, the generated data can be processed before storing. In other implementations, the generated data can be wirelessly transmitted to an external computer system for further analysis.

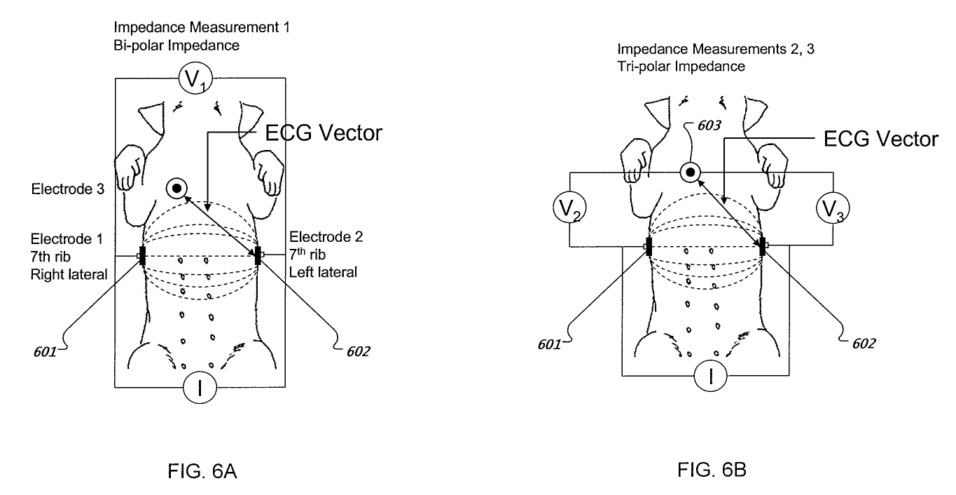

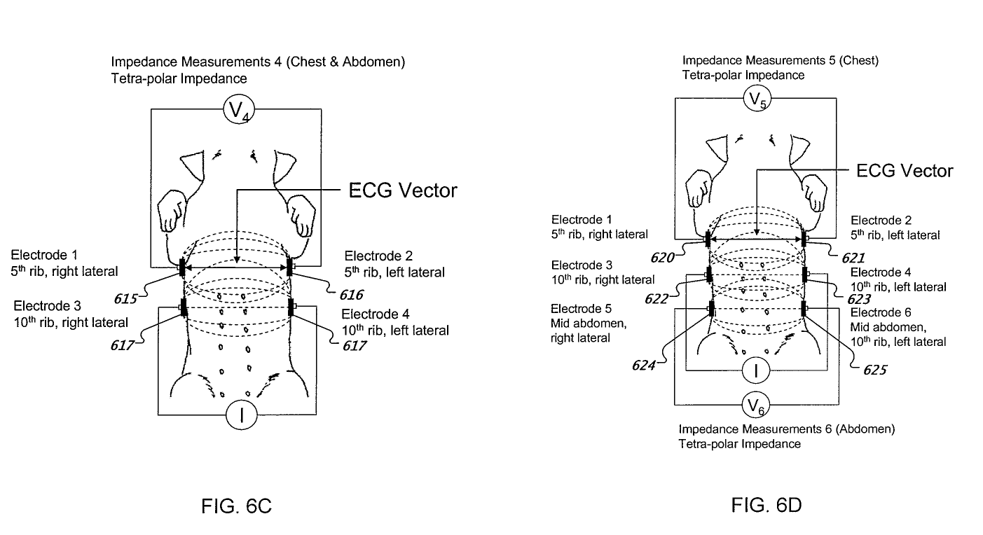

FIGS. 5A-B are provided as an anatomical reference for FIGS. 6A-D. In particular, FIGS. 5A-B illustrates relationships between the internal organs-particularly the heart, lungs and diaphragm-and the relationships between those organs and specific ribs. In placing lead wires having current and voltage electrodes, one may find the ribs to be particularly helpful external reference points, and specific ribs are mentioned as such with respect to the following figures.

FIGS. 6A-D illustrate exemplary implementations of electrode arrangements that can be used to monitor ECG and respiration for an animal, for example as part of one or more of the systems 100, 201 and 300 described above.

Turning to FIG. 6A, an example bipolar electrode arrangement is illustrated, in which electrodes 601 and 602 are disposed at left and right lateral points, near the 7^{th} rib. As shown, the current signal is provided by the same electrodes 601 and 602 from which the voltage signal V1 is measured. A lead field corresponding to an example current signal is shown, and in some implementations, the lead field corresponding to the voltage is substantially coextensive with the current lead field. The electrode arrangement shown in FIG. 6A may be particularly suitable for measuring ECG and impedance values of an animal in a single-channel configuration.

FIG. 6B illustrates a tripolar configuration, in which a current signal can again be provided by left and right lateral electrodes 601 and 602, and a third electrode 603 can be employed to obtain two different voltage signals, V2 and V3. As depicted in one example in FIG. 6B, the third electrode 603 can be disposed medially in a right pectoral region. By moving this electrode with respect to electrodes 601 and 602, one may be able to affect the relative magnitudes of respiration and cardiac components in the voltage signals V2 and V3.

FIG. 6C illustrates an example tetrapolar configuration in which two electrodes 615 and 616 are disposed in the chest or thorax region—specifically, in this example, at left and right lateral points about in line with the 5^{th} rib; two other electrodes 617 and 618 are shown in left and right lateral positions about in line with the 10^{th} rib. As depicted, a current signal can be provided by electrodes 617 and 618, and a voltage V4 signal can be measured by electrodes 615 and 616. In such an implementation, the voltage signal V4 may include a respiration component that corresponds with the middle lobes of the lungs. In other implementations, the voltage and current signals could be reversed (e.g., to obtain a voltage measurement that is more influenced by the diaphragm.

FIG. 6D illustrates an example six-electrode configuration in which pairs of electrodes are placed left laterally and right laterally, about in line with the 4^{th} through 5^{th} ribs (electrodes 620 and 621), 6^{th} through 10^{th} ribs (electrodes 622 and 623) and 11^{th} rib to mid-abdomen (electrodes 624 and 625). As depicted, a current signal can be provided at electrodes 622 and 623, such that the current signal extends in both cranial and caudal directions from the electrodes 622 and 623. Two voltage signals V5 and V6 can be obtained. From the cranial electrodes 620 and 621, a voltage signal V5 can be obtained that may correspond to the majority of the lungs mass. From the caudal electrodes 624 and 625, a voltage signal V6 can be obtained that may correspond to the diaphragm and abdominal region.

In each of the example configurations described above, each electrode could be disposed on a single, distinct lead wire, or multiple electrodes could be disposed on one lead wire. Different arrangements may be suitable for different contexts. For example, distinct lead wires for each electrode provide more flexibility in positioning. On the other hand, disposing multiple electrodes on a single lead wire (e.g., in the example of FIG. 6D, one lead wire for electrodes 620, 622 and 624, and a second lead wire for electrodes 621, 623 and 625) may make implantation of the electrodes more straightforward; and for multiple test subjects that are approximately the same size, a predetermined, fixed spacing between electrodes may result in more uniformity of measurements between test subjects. Electrodes that are individually disposed on lead wires may be especially appropriate for larger animals, while multi-electrode lead wires may be important to minimizing implantation trauma in smaller animals.

Whatever electrode configuration is employed, a voltage signal that is obtained from the configuration can be processed in various ways, some of which are described in detail in application Ser. No. 11/933,872, filed Nov. 1, 2007, by Moon et al. For example, an impedance signal may be determined at the processing and control device 106 (FIG. 1) from the current and voltage signals. Cardiac and respiration components of this signal may be filtered as necessary, for example using the filtering circuitry 316 (FIG. 3), and appropriate values may be stored in the processing and control device 106 for later retrieval, or transmitted in real time a system external to the animal monitoring system 100 (FIG. 1). As another example, values representative of the voltage signal (e.g., discrete, time-ordered values) may be stored or transmitted, and impedance may be calculated outside the animal monitoring system 100. Numerous data processing actions are possible and contemplated.

A number of embodiments have been described. Nevertheless, it will be understood that various modifications may be made without departing from the spirit and scope of this disclosure. Accordingly, other embodiments are within the scope of the following claims.

What is claimed is:

1. An ambulatory animal monitoring system comprising:
a wearable structure constructed to be worn about a body of a non-human animal to be monitored;
a plurality of electrical signal conduits each associated with the wearable structure and each connectable to a different one of a plurality of surface electrode components; and
processing and control device adapted to be worn with the wearable structure, the processing and control device comprising a) an ECG monitoring component that generates data indicative of an ECG signal over time by measuring the ECG signal of the animal using surface electrode components connected to the plurality of electrical signal conduits; and b) an impedance level monitoring component that generates data indicative of electrical impedance levels of the animal over time by i) injecting current between at least two surface electrode components connected to the plurality of electrical signal conduits, and ii) measuring a resulting voltage level between at least two surface electrode components connected to the plurality of electrical signal conduits, at least one of the surface electrode components between which the current is injected not being used for the measuring of the resulting voltage.

2. The animal monitoring system of claim 1, wherein the plurality of electrical signal conduits comprise at least three electrical signal conduits.

3. The animal monitoring system of claim 1, wherein at least one surface electrode component whose output is connected to the plurality of electrical signal conduits is used both in measuring the ECG signal and in measuring the electrical impedance level.

4. The animal monitoring system of claim 1, wherein the impedance measurement circuitry comprises two channels of impedance measurement circuitry for measuring over time an electrical impedance level between two different sets of surface electrode components whose outputs are connected to the plurality of electrical signal conduits, so as to measure over time an electrical impedance level across two different portions of the animal's body.

5. The animal monitoring system of claim 2, wherein the plurality of electrical signal conduits comprise at least six electrical signal conduits (ESC1 through ESC6) for using at least six surface electrode components (SEC1 through SEC6) with the animal monitoring system.

6. The animal monitoring system of claim 5, wherein at least electrical signal conduits ESC1, ESC2, ESC4 and ESC5 are used for electrical impedance measurements; and at least electrical signal conduits ESC2 and ESC4 are used for ECG measurement.

7. The animal monitoring system of claim 6, wherein electrical signal conduits ESC3 and ESC 6 are also used for impedance measurements, with electrical signal conduits ESC1, ESC2, ESC4 and ESC5 being used for a first channel of electrical impedance measurements, and electrical signal conduits ESC2, ESC3, ESC5 and ESC6 being used for a second channel of electrical impedance measurements.

8. The animal monitoring system of claim 1, further comprising a telemetry component adapted to be worn with the wearable structure, the telemetry component for wirelessly communicating to another device the generated data indicative of an ECG signal over time and the generated data indicative of electrical impedance levels of the animal over time.

9. The animal monitoring system of claim 1, wherein the system is configured such that when the system is placed on an animal to be monitored, the system does not restrict ambulatory movement of the animal by way of tethering the animal to stationary equipment.

10. The animal monitoring system of claim 1, wherein the processing and control device is adapted to determine from the electrical impedance measures whether a placement of an electrode component on the animal is unsatisfactory.

11. The animal monitoring system of claim 10, wherein the processing and control device is further adapted to perform the ECG sensing and the impedance measuring using electrode components other than an electrode component whose placement has determined to be unsatisfactory.

12. The animal monitoring system of claim 10, wherein the processing and control device is further adapted to produce a signal indicative of an electrode component having an unsatifactory placement.

13. The animal monitoring system of claim 1, wherein the impedance level monitoring component generates the data indicative of electrical impedance levels such that the data is configured to be used in monitoring animal respiration.

14. An animal monitoring system comprising:
wearable componentry comprising:
a wearable structure constructed to be worn about a body of a non-human animal to be monitored;
a plurality of electrical signal conduits each associated with the wearable structure and connectable to a different one of a plurality of surface electrode components;
processing and control apparatus adapted to be worn with the wearable structure, the processing and control apparatus comprising a) an ECG monitoring component that generates data indicative of an ECG signal over time by measuring the ECG signal of the animal using surface electrode components connected to the plurality of electrical signal conduits; and b) an impedance level monitoring component that generates data indicative of electrical impedance levels of the animal over time by i) injecting current between at least two surface electrode components connected to the plurality of electrical signal conduits, and ii) measuring a resulting voltage level between at least two surface electrode components connected to the plurality of electrical signal conduits, at least one of the surface electrode components between which the current is injected not being used for the measuring of the resulting voltage; and
a telemetry component adapted to be worn with the wearable structure, the telemetry component for wirelessly communicating the generated data indicative of an ECG signal over time and the generated data indicative of electrical impedance levels of the animal over time; and
receiving and processing apparatus comprising a wireless receiver that receives the generated data wirelessly transmitted from the telemetry component of the wearable componentry, and an analysis module for analyzing ECG and respiration of the animal based on the generated data.
